(19) United States
(12) Patent Application Publication
(10) Pub. No.: US 2006/0141499 A1
(43) Pub. Date: Jun. 29, 2006

(54) Methods of determining human egg competency

(76) Inventors: Geoffrey Sher, Las Vegas, NV (US); Levent Keskintepe, Las Vegas, NV (US)

Correspondence Address: MCDERMOTT WILL & EMERY LLP, 600 13TH STREET, N.W., WASHINGTON, DC, 20005-3096, US

(21) Appl. No.: 11/280,855

(22) Filed: Nov. 17, 2005

Related U.S. Application Data
(60) Provisional application No. 60/628,125, filed on Nov. 17, 2004.
(60) Provisional application No. 60/628,126, filed on Nov. 17, 2004.

(51) Int. Cl. C12Q 1/68; C12N 15/00

(52) U.S. Cl. 435/6; 800/21

(57) ABSTRACT

The inventors have discovered inter alia, that when the chromosome complement of a first polar body (PB-I) is normal, it alone is in more than 80% of cases sufficient to predict that an embryo which has developed from an oocyte associated with the PB-I is also normal and competent. As such, the invention relates to methods of identifying competent oocytes and embryos suitable for use during in vitro fertilization.

### PRIORITY

The application claims the benefit of U.S. Ser. Nos. 60/628,125 filed Nov. 17, 2004 and 60/628,126 filed Nov. 17, 2004.

### BACKGROUND OF THE INVENTION

1. Field of the Invention

This invention relates to the field of human reproductive medicine. More specifically, it relates to methods of determining the competency of human eggs and embryos.

2. Background

There are unique challenges to having successful healthy pregnancies after the age of 35. Specifically, a woman's egg quality—a major variable in her fertility—begins to decline in her late twenties. According to the American Society for Reproductive Medicine (ASRM), a woman over age 40 has only a 5 percent chance or less of becoming pregnant naturally in any one month. Furthermore, the risk of chromosomal abnormalities in newborns increases with the age of the woman's egg, growing to one in 66 at age 40 versus one in 385 at age 30.

As such, most women in their 40's are not able to successfully carry a “natural” pregnancy to term. However, when these women use donor eggs from younger women, they achieve the same pregnancy success rates as women in their 20's. This demonstrates that the primary cause of infertility and miscarriages in older women is the age (quality) of the egg, not the uterus.

Without the ability to accurately differentiate between competent oocytes (i.e., those that can develop into an embryo which is capable of implantation in a healthy female and resulting in a viable pregnancy) and incompetent oocytes, in vitro fertilization (IVF) specialists will inevitably, unwittingly, and repeatedly process incompetent oocytes and transfer incompetent embryos to women thereby compromising the ability to initiate a successful pregnancy. The converse is also true, namely that the ability to accurately and reliably select competent oocytes and embryos in an IVF program would: (i) lead to markedly improved IVF success rates; (ii) profoundly reduce the incidence of multiple pregnancies; (iii) dramatically reduce the cost necessary to achieve a viable pregnancy; and (iv) reduce reproductive healthcare costs.

Oocyte competency depends in large part on the genetic make up of the oocyte, i.e., its ploidy. An oocyte can either be genetically health, i.e., euploid, or genetically deficient, i.e., aneuploid. Human beings have an inordinately high incidence of spontaneous germ cell aneuploidy due to abnormal crossing over during meiotic recombination in prophase I (Gutiérrez-Mateo et al. Hum. Reprod. 2004; 19: 2859-2868, Lenzi et al., 2005). Reportedly >50% of IVF-harvested oocytes as well as the reciprocal embryos have been found to be aneuploid with the incidence increasing with advancing age and in morphologically abnormal embryos.

To date, all of the techniques used for studying aneuploidy in oocytes have been based on the spreading of the chromosome material onto slides, followed by methods such as: banding techniques, fluorescence in situ hybridization (FISH) for up to nine chromosomes, spectral karyotyping (SKY) or multicolour fluorescence in situ hybridization (m-FISH). The dependence on spreading of chromosomes has led to problems not only with overlapping chromosomes, chromosome morphology and artefactual loss of chromosomes during spreading, but also because of the difficulty of obtaining chromosome banding in metaphase II (MII) chromosomes to allow identification of specific chromosome aneuploidies. FISH studies have an extra limitation, as less than a half of the whole karyotype can be analyzed because accuracy per probe is reduced when large numbers of probes are combined.

Magli et al (Hum Reprod. May 2004;19(5):1163-9) performed 8-probe FISH on the first polar bodies (PB-I) as well as single blastomeres from reciprocal embryos obtained from 113 IVF cycles in an attempt to increase the quantity of DNA available for genetic analysis. They concluded that the biopsy procedures did not compromise subsequent embryo development or implantation potential and accordingly could be used for making a combined diagnosis of aneuploidy and single-gene disorders in preimplantation embryos generated by couples at high reproductive risk.

Whole genome amplification (WGA) and comparative genomic hybridization (CGH) have previously been used to identify the presence of genomic imbalance in embryonic cells during pre-implantation genetic diagnosis (PGD). CGH is a molecular cytogenetic technique that allows the analysis of the full set of chromosomes in single cells. CGH, as a DNA-based method which does not involve cell fixation, may overcome these limitations by analyzing the whole set of chromosomes and giving a more accurate and reliable evaluation of the aneuploidy rate (both hyperhaploidy and hypohaploidy). CGH has been used to the study of numerical and structural abnormalities of single blastomeres from disaggregated 3-day-old human embryos. (Voullaire et al., Hum Genet. February 2000;106(2):210-7). Gutierrez-Mateo et al., (Hum Reprod. September 2004;19(9):2118-25) analyzed by CGH both, a large number of first polar bodies (PB-Is) and metaphase II (MII) oocytes and found an aneuploidy rate of 48%. A higher number of chromosome abnormalities was detected in the oocytes from older donors. Moreover, about a third of the PB-I-MII oocyte doublets diagnosed as aneuploid by CGH would have been misdiagnosed as normal if FISH with nine chromosome probes had been used. As such, CGH is a reliable analytic technique for PB-I analysis for detecting oocyte chromosomal abnormality in addition to unbalanced segregations.

The development of the ovarian follicle and the production of a competent oocyte involve a series of developmental events which culminate at ovulation. These events are controlled by hormones of pituitary and local ovarian origin, and by secretions from other organs. The resulting intra-follicular environment has profound effects on follicle maturation, oocyte quality and embryo survival.

Costa et al. (Braz J Med Biol Res. November 2004;37(11):1747-55) examined the association between follicular fluid (FF) steroid concentration and oocyte maturity and fertilization rates. Progesterone, estradiol (E2), estrone, androstenedione, and testosterone were measured in the FF of a number of infertile women following human chorionic gonadotropin induction. E2 and testosterone levels were significantly higher in FF containing immature oocytes than in FF containing mature oocytes. Progesterone, androstenedione and estrone levels were not significantly different between mature and immature oocytes. However, the authors observed a significant increase in progesterone/testosterone, progesterone/E2 and E2/testosterone ratios in FF containing mature oocytes, suggesting a reduction in conversion of C21 to C19, but not in aromatase activity. The overall fertility rate was 61% but the authors observed no correlation between the steroid levels or their ratios and the fertilization rates. The authors concluded that E2 and testosterone levels in FF may be used as a predictive parameter of oocyte maturity, but not for the in vitro fertilization rate.

There are two subtypes of T helper cells (Th1 and Th2), at the embryo-decidual interphase (Jurisicova et al., Mol Hum Reprod. February 1996;2(2):93-8; Proc Natl Acad Sci U S A. Jan. 9, 1996;93(1):161-5). Th1 predominates in the non-pregnant state and expresses interferon (IFN) gamma and tumor necrosis factor (TNF) alpha, the cytokines predominantly involved in cellular immunity, delayed hypersensitivity, tissue injury in infection and autoimmune disease. Th2 helper cells secrete IL-1, IL-4, IL-5, IL-6, IL-9, IL-10, and IL-13, cytokines that are involved in antibody production. Th2 response down regulates the Th1 response and vice versa. The balance between certain Th1 and Th2 cytokines largely predominates whether an induced shift towards Th2 during pregnancy establishes and perhaps co-ordinates a cytokine network that protects the developing embryo from rejection by the maternal immune system.

Cryopreserving eggs can effectively slow down a woman's biological clock by capturing a woman's healthy “young” eggs and freezing them for use in the future. Until very recently, egg freezing, or oocyte cryopreservation, was carried out only in carefully controlled research settings and was available only to young women facing chemotherapy or suffering from illnesses that might make them infertile.

Egg freezing has yet to become widespread because, while sperm and embryos freeze fairly easily, eggs are much more fragile. Egg cells contain a lot of water and as a result, ice crystals can form that may damage the egg's structure. Harvesting eggs for freezing involves a process similar to that of in vitro fertilization: For about a month, a woman must give herself hormone injections to stimulate the ovaries to produce more than one egg. Typically, a cycle of fertility drugs will produce about 12 to 15 eggs, which are then drawn into a needle. The eggs are frozen using a cryoprotectant formula that helps dehydrate the watery eggs so that they can be safely frozen without forming damaging ice crystals. When a woman is ready to use her eggs, a thawing formulation reverses the process, rehydrating the eggs back to their original state.

Less than 10% of oocytes currently being cryopreserved actually survive the subsequent thaw in a condition that allows for successful fertilization and pregnancy generation. This low yield has in the past, almost invariably been attributed to the lack of availability of optimal freezing techniques, all but ignoring the fact that most of the oocytes cryopreserved have been aneuploid. As stated above, the increasing age of the patient is associated with a decline in egg quality. The inventors note that it is more commonly older females (>35 years) who, fearful of the inevitable decline in fertility with advancing age, express interest in cryopreserving their eggs.

Given the cost and labor-intensive and already unpredictable nature of cryopreservation and in vitro fertilization, it is imperative that only a woman's highest quality oocytes are frozen. However, in lieu of a methodology for identifying high quality oocytes prior to cryopreservation, most fresh and frozen oocytes (irrespective of whether they are euploid or aneuploid) are blindly fertilized and those that develop to the embryo stage will be transferred if they appear to have a good chance of implantation. This is the usual procedure and it risks and often generates unwanted multiple pregnancies.

The presumption has always been that it is better to transfer healthy embryos into the uterus sooner rather than later once the best ones for transfer have been identified. It is against this background that attention has been focused on the transfer of more-developed embryos (blastocysts). The transfer of good-quality blastocysts is associated with a high rate of pregnancy, but it carries with it a concomitant risk of high-order multiple pregnancies (triplets or greater) unless fewer blastocysts are transferred because they are considered more likely to implant. Implantation of an embryo with a high embryonic grade significantly increases the chances that it will successfully develop into a healthy fetus and normal baby.

In the past, approaches aimed at identifying the competent embryos for transfer focused on: (i) morphological assessment prior to embryo transfer (ET), and (ii) Preimplantation Genetic Diagnosis (PGD) on one or more blastomeres.

A graduated embryo scoring (GES) system has recently been introduced, in which an embryo is separately cultured in its own well, allowing for sequential microscopic morphological assessment of developmental criteria. (Fisch et al., Fertil Steril. December 2003;80(6):1352-8). A score is then assigned to each embryo on day 3 post-oocyte retrieval. It was possible to demonstrate that embryos that score 70 out of a possible 100 allotted points have the greatest potential to implant after being transferred to the uterus and/or survive the blastocyst stage is maintained in culture for 2-3 additional days.

Morphological embryo and blastocyst evaluations, while furnishing clues that can enhance proficiency in choosing the embryos for transfer are severely flawed in their ability to provide sturdy evidence of embryo polidy. This is pressing because there is some evidence that suggests that there is a negative selection against some chromosome abnormalities during the first stages of embryonic development. This may explain the fact that the rate of aneuploidies in cleavage-stage embryos is much higher than that found in spontaneous abortions and liveborns.

PGD with commercially available fluorescence in situ hybridization (FISH), similarly lacks sensitivity and specificity when it comes to assessing embryo ploidy since, available FISH probes can only examine 8-10 of the 23 human chromosome pairs. Thus, many chromosome pairs cannot be examined for numerical abnormalities (aneuploidy). In fact, it has been reported that even when commercially available FISH reveals that these 8-10 targeted chromosome pairs are intact, there remains a 40%-50% chance that aneuploidy affecting the remaining chromosome pairs, might still exist.

Current developments and discoveries are changing the way IVF is performed by bringing IVF practitioners much closer to the long-awaited objective of “one embryo, one healthy baby.” The use of biochemical and genetic markers of oocyte and embryo quality could also provide researchers as well as the pharmaceutical industry with a method that would help in the development of new and more efficacious fertility drugs that produce fewer side effects with reduced risk to patients.

The invention provides these and other advantages, as will be apparent to those skilled in the art based on the disclosure hereunder. All references and documents cited herein are incorporated by reference in their entirety.

### SUMMARY OF THE INVENTION

One aspect of the invention relates to a method of selecting a euploid oocyte comprising: harvesting at least one oocyte from a female; isolating a first polar body associated with the at least one oocyte; analyzing the genetic euploid of the first polar body to obtain a genetic analysis parameter; identifying a competent oocyte by determining if the genetic analysis parameter is indicative of oocyte euploidy; and selecting the euploid oocyte.

In one embodiment of this aspect of the invention, the the analyzing is done by comparative genomic hybridization (CGH). In another embodiment the genetic analysis parameter indicative of oocyte euploidy is from about 0.8:1 to about 1.2:1. In yet another embodiment, the genetic analysis parameter indicative of oocyte euploidy is from about 0.9:1 to about 1.1:1. In still another embodiment, the genetic analysis parameter indicative of oocyte euploidy is about 1:1. In a further embodiment the selected euploid oocyte is frozen and stored for a period of time. In yet a further embodiment the selected euploid oocyte is fertilized with euploid sperm. In still a further embodiment the fertilized oocyte is cultured to obtain a euploid embryo. In another embodiment the the embryo is frozen and stored for a period of time. In yet another embodiment the embryo is competent. In still a further embodiment the embryo is transferred to a recipient female.

Another aspect of the invention relates to a method of determining the competence of an embryo comprising: harvesting at least one oocyte from a female; isolating a first polar body associated with the at least one oocyte; analyzing the genome of the first polar body to obtain a genetic analysis parameter; correlating the genetic analysis parameter with the ploidy of the oocyte; selecting and fertilizing the oocyte with euploid sperm to obtain an embryo; and determining that the embryo is competent if the genetic analysis parameter of the first polar body associated with the oocyte from which the embryo was derived, is indicative of euploidy.

In one embodiment of this aspect of the invention, the the analyzing is done by comparative genomic hybridization (CGH). In another embodiment the genetic analysis parameter indicative of oocyte euploidy is from about 0.8:1 to about 1.2:1. In yet another embodiment, the genetic analysis parameter indicative of oocyte euploidy is from about 0.9:1 to about 1.1:1. In still another embodiment, the genetic analysis parameter indicative of oocyte euploidy is about 1:1. In a further embodiment the selected euploid oocyte is frozen and stored for a period of time prior to fertilization.

Another aspect of the invention relates to a method of impregnating a recipient female comprising: harvesting at least one oocyte from a donor female; isolating a first polar body associated with the at least one oocyte; analyzing the genome of the first polar body to obtain a genetic analysis parameter; correlating the genetic analysis parameter with the euploidy of the oocyte; selecting and fertilizing the at least one oocyte with euploid sperm to a zygote; culturing the zygote to obtain an embryo; determining that the embryo is competent if the genetic analysis parameter of the first polar body associated with the oocyte from which the embryo was derived, is indicative of euploidy and transferring the embryo to the female recipient if it was determined to be competent.

In one embodiment of this aspect of the invention, the recipient female and the donor female are the same individual. In another they are different. In another embodiment the analyzing is done by comparative genomic hybridization (CGH). In another embodiment the genetic analysis parameter indicative of oocyte euploidy is from about 0.8:1 to about 1.2:1. In yet another embodiment, the genetic analysis parameter indicative of oocyte euploidy is from about 0.9:1 to about 1.1:1. In still another embodiment, the genetic analysis parameter indicative of oocyte euploidy is about 1:1. In a further embodiment the oocyte is frozen and thawed prior to fertilization. In still a further embodiment the embryo is frozen, stored for a period of time and unfrozen prior to transfer to recipient female, if it was determined to be competent.

Another aspect of the invention relates to a method of identifying a candidate fertility drug comprising: harvesting a first plurality of oocytes from a female; isolating a first polar body associated with each of the plurality of oocytes; analyzing the genome of each first polar body to obtain a genetic analysis parameter for each ooctye; identifying a euploid oocyte by determining if its genetic analysis parameter is indicative of oocyte euploidy; comparing the number of euploid oocytes to the number of oocytes to obtain a baseline euploid ratio; administering to the female a test compound; harvesting a second plurality of oocytes from the female; analyzing the genome of first polar bodies associated with each of the second plurality of oocytes; identifying euploid oocytes of the second plurality of oocytes by determining if the genetic analysis parameters are indicative of oocyte euploidy; comparing the number of euploid oocytes to the number of oocytes to obtain a test euploid ratio; and identifying a candidate fertility drug if the administration correlates with an increase in the test euploid ratio compared to the baseline euploid ratio.

In one embodiment of this aspect of the invention, the female is a human. In another embodiment the analyzing is done by comparative genomic hybridization (CGH). In another embodiment the genetic analysis parameter indicative of oocyte euploidy is from about 0.8:1 to about 1.2:1. In yet another embodiment, the genetic analysis parameter indicative of oocyte euploidy is from about 0.9:1 to about 1.1:1. In still another embodiment, the genetic analysis parameter indicative of oocyte euploidy is about 1:1.

Another aspect of the invention relates to a method of determining the efficacy of a fertility drug therapeutic regimen on a female comprising: harvesting a first plurality of oocytes from the female; isolating a plurality of first polar bodies associated with each oocyte; analyzing the genetic component of each first polar body to obtain a genetic analysis parameter for each ooctye; identifying a euploid oocyte by determining if the genetic analysis parameters are indicative of oocyte euploidy; comparing the number of euploid oocytes to the number of oocytes to obtain a baseline euploidy ratio; administering to the female a fertility drug therapeutic regimen; harvesting a second plurality of oocytes from the female; analyzing the genetic component of each first polar body to obtain a test genetic analysis parameter for each ooctye harvested in the second plurality of oocytes; identifying euploid oocytes in the second plurality of oocytes by determining if the test genetic analysis parameters are indicative of oocyte euploidy; comparing the number of euploid oocytes to the number of oocytes to obtain a test euploid ratio; and determining that the fertility drug therapeutic regimen is effective when the administration of the fertility drug therapeutic regimen correlates with an increase in the test euploid ratio compared to the baseline euploid ratio.

In one embodiment of this aspect of the invention, the female is a human. In another embodiment the analyzing is done by comparative genomic hybridization (CGH). In another embodiment the genetic analysis parameter indicative of oocyte euploidy is from about 0.8:1 to about 1.2:1. In yet another embodiment, the genetic analysis parameter indicative of oocyte euploidy is from about 0.9:1 to about 1.1:1. In still another embodiment, the genetic analysis parameter indicative of oocyte euploidy is about 1:1.

Another aspect of the invention relates to a method of determining oocyte competency comprising: inducing ovulatation in at least one female; harvesting at least one oocyte and aspirating a matching follicular fluid sample from each of the at least one female; determining at least one of the following follicular fluid parameters: the concentration of androgens in the follicular fluid sample; and/or the balance of the concentration of Th1 and Th2 cytokines in the follicular fluid sample; grading the at least one oocyte to obtain an oocytes grade of “1,” “2,” or “3”; removing a polar body associated with the at least one oocytes and obtaining at least one genetic analysis parameter by performing: an analysis of the short arm of chromosome 6, to obtain a chromosome 6 analysis parameter; and/or a comparative genomic hybridization analysis, to obtain a comparative genomic hybridization parameter; and fertilizing the at least one oocyte to obtain an embryo; grading the embryo according to the graduated embryo scoring system to obtain an embryo grade; delivering the embryo to a female to facilitate embryonic development; creating a database wherein the competency of the embryo is correlated with: at least one follicular fluid parameter; the oocyte grade of the embryo; at least one genetic analysis parameter; and/or the embryo grade; harvesting at least one test oocyte and aspirating a test matching follicular fluid sample from at least one test female; and querying the database to determine if the test follicular fluid sample correlates with competency embryonic.

In one embodiment of this aspect of the invention the androgens are testosterone and androstenedione. In another embodiment, the concentration of androgens in the follicular fluid sample and the balance of the concentration of certain Th1 and Th2 cytokines in the follicular fluid sample are measured. In yet another embodiment, the Th1 cytokines are a combination of cytokines selected from the group consisting of interferon gamma, tumor necrosis factor (TNF) alpha, IL-2 and IL-3, and the Th2 cytokines are a combination of cytokines selected from the group consisting IL-1, IL-4, IL-5, IL-6, IL-9, IL-10, and IL-13. In a further embodiment the analysis of the short arm of chromosome 6 is done by fluorescence in situ hybridization (FSH). In yet a further embodiment, the analysis, the comparative genomic hybridization parameter is indicative of a loss or a gain of DNA with respect to a reference DNA sample.

Another aspect of the invention relates to a method of creating a database for determining whether to cryopreserve an oocyte comprising: inducing ovulatation in at least one female; harvesting at least one oocyte and aspirating a matching follicular fluid sample from the at least one female; determining at least one of the following follicular fluid parameters; the concentration of androgens in the follicular fluid sample; and/or the balance of the concentration of certain Th1 and Th2 cytokines in the follicular fluid sample; grading the at least one oocyte to obtain an oocyte grade of“1,” “2,” or “3”; removing a polar body associated with the at least one oocyte and obtaining at least one genetic analysis parameter by performing an analysis of the short arm of chromosome 6, to obtain a chromosome 6 analysis parameter; and/or a comparative genomic hybridization analysis, to obtain a comparative genomic hybridization parameter; freezing the oocyte; thawing the oocyte; fertilizing the at least one oocytes to obtain an embryo; grading the embryo according to the graduated embryo scoring system to obtain an embryo grade; delivering the embryo to a female to facilitate embryonic development; creating a database wherein the development the embryo is correlated with the at least one follicular fluid parameter; the oocyte grade of the embryo; at least one genetic analysis parameter; and/or the embryo grade.

In one embodiment, the androgens are testosterone and androstenedione. In another embodiment, the concentration of androgens in the follicular fluid sample and the balance of the concentration of Th1 and Th2 cytokines in the follicular fluid sample are measured. In another embodiment, the Th1 cytokines are a combination of cytokines selected from the group consisting of interferon (IFN) gamma, tumor necrosis factor (TNF) alpha, IL-2 and IL-3, and the Th2 cytokines are combination of cytokines selected from the group consisting IL-1, IL-4, IL-5, IL-6, IL-9, IL-10, and IL-13. In yet a further embodiment, the analysis of the short arm of chromosome 6 is done by fluorescence in situ hybridization (FSH). In yet another embodiment, the comparative genomic hybridization parameter is indicative of a loss or a gain of DNA with respect to a reference DNA sample.

Another aspect of the invention relates to a method of creating an egg bank comprising: inducing ovulation in a female patient; harvesting at least one oocyte and aspirating a matching follicular fluid sample from the patient; determining at least one of the following follicular fluid parameters: the concentration of androgens in the follicular fluid sample; and/or the balance of the concentration of Th1 and Th2 cytokines in the follicular fluid sample; querying a database to determine if the at least one follicular fluid parameter correlates with oocyte competency; selecting at least one oocyte whose follicular fluid parameters correlate with competency; and freezing the at least one oocyte whose follicular fluid parameters correlate with competency, to form an egg bank.

In one embodiment of this aspect of the invention, the database correlates the development of an embryo with at least one follicular fluid parameter; the oocyte grade of the embryo; at least one genetic analysis parameter; and/or the embryo grade.

In another embodiment the androgens are testosterone and androstenedione. In yet a further embodiment, the concentration of androgens in the follicular fluid sample and the balance of the concentration of Th1 and Th2 cytokines in the follicular fluid sample are determined. In yet another embodiment, the Th1 cytokines are combination of cytokines selected from the group consisting of interferon (IFN) gamma, tumor necrosis factor (TNF) alpha, IL-2 and IL-3, and the Th2 cytokines are combination of cytokines selected from the group consisting IL-1, IL-4, IL-5, IL-6, IL-9, IL-10, and IL-13. In another embodiment, the at least one genetic analysis parameter comprises analysis of the short arm of chromosome 6 by fluorescence in situ hybridization (FISH). In another embodiment, the at least one genetic analysis parameter comprises analysis by comparative genomic hybridization parameter and is indicative of a loss or a gain of DNA with respect to a reference DNA sample. In still another embodiment, oocytes whose follicular fluid parameters correlate with competency are added to the egg bank. In a further embodiment, additional oocytes are derived from the same female. In yet a another embodiment, the additional oocytes are derived from a different female.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail, in order not to unnecessarily obscure the present invention.

### DETAILED DESCRIPTION OF THE INVENTION

Before describing the invention in greater detail the following definitions are set forth to illustrate and define the meaning and scope of the terms used to describe the invention herein:

“Ploidy” as used herein refers to the number of copies of the basic number of chromosomes for a particular cell type. The number of basic sets of chromosomes in an organism is called the monoploid number (X). In humans, most cells are diploid (containing one set of chromosomes from each parent), though sex cells (sperm and oocytes) are haploid.

“Euploidy” or “genetically normal” as used herein is the condition of having a normal number of structurally normal chromosomes. Euploid human females have 46 chromosomes (22 pairs of autosomes and two X chromosomes). Euploid human males have 46 chromosomes (22 pairs of autosomes and an X chromosome and a Y chromosome). Euploid, i.e., genetically normal, human oocytes and contain 22 autosomes and an X chromosome. Incompetent oocytes are usually aneuploid. Euploid, i.e., genetically normal, human sper and contain 22 autosomes and either an X or Y chromosome.

“Aneuploidy” is the condition of having less than or more than the normal diploid number of chromosomes, and is the most frequently observed type of cytogenetic abnormality. Aneuploidy also occurs when a cell contains an abnormal or non-integer ploidy number. This may lead to problems in cell development. The two most commonly observed forms of aneuploidy are monosomy and trisomy. Monosomy is the lack of one of a pair of chromosomes. For example, an individual having only one chromosome 6 is said to have monosomy 6. A common monosomy seen in many species is X chromosome monosomy, also known as Turner's syndrome. Monosomy is most commonly lethal during prenatal development. Trisomy is having three chromosomes of a particular type. A common autosomal trisomy in humans is Down syndrome, or trisomy 21, in which a person or coceptus has three instead of the normal two chromosome 21s. Trisomy is a specific instance of polysomy, a more general term that indicates having more than two of any given chromosome. A chromosome deletion occurs when the chromosome breaks and a piece is lost. This of course involves loss of genetic information and results in what could be considered “partial monosomy” for that chromosome. A related abnormality is a chromosome inversion. In this case, a break or breaks occur and that fragment of chromosome is inverted and rejoined rather than being lost. Inversions are thus rearrangements that do not involve loss of genetic material and, unless the breakpoints disrupt an important gene, individuals carrying inversions have a normal phenotype.

Aneuploidy is also recognized as a small deviation from euploidy for the simple reason that major deviations are rarely compatible with survival, and such individuals usually die prenatally. Therefore, as used herein, aneuploidy refers to any deviation from euploidy, notwithstanding the terms used in the art to connote conditions in which only a small number of chromosomes are missing or added.

For purposes of this invention and specification, the term “competence” as applied to the oocyte means that: (1) its karyotype is euploid and (2) following fertilization with euploid sperm it spawns a euploid embryo that, following transfer to a receptive uterus, e.g., one lacking implantation dysfunction, would have a great likelihood of resulting in the development of a euploid fetus and baby.

Oocyte euploidy is strongly indicative of oocyte competence because all competent oocytes are euploid. However, because of non-genetic factors not all euploid oocytes are necessarily competent. The skilled artisan will recognize that not all euploid oocytes necessarily develop into an embryo upon fertilization. For example, there may be deficiencies with regard to the amount or nature of maternal components contributed to the oocyte or the oocyte may have been subject to excessive physical/physiological trauma during retrieval, freezing, thawing and/or ICSI. As such, whether an oocyte fertilized with euploid sperm develops into an embryo depends on three factors: 1) genetics; 2) the cellular make up of the oocyte; and 3) environmental factors.

For purposes of this invention and specification, the term “competence” as applied to the embryo expresses that: (1) its karyotype is euploid: (2) it was derived from a competent oocyte, and (3) following transfer to a female lacking health problems such as implantation dysfunction, it would spawn a euploid fetus and baby.

Whereas not all embryos are euploid, all competent embryos are. For example, when an oocyte is fertilized with healthy sperm and develops into a embryo, the genetic, cellular and environmental factors were sufficient to allow for the development of an embryo. However, the genetic component necessary to make an embryo is not sufficient to make a competent embryo, i.e., one that will result in a euploid fetus. There may be genetic deficiencies that will allow for the development of an embryo, but will block the development of a normal fetus and result in miscarriage. Accordingly, whether or not an embryo, upon delivery to a normal female (e.g., a female lacking any form of implantation dysfunction), is capable of resulting in an the development of a euploid fetus and baby depends on the genetic complement of the embryo.

The term “inducing ovulation in at least one female” refers to providing a female with agents that elicit the full development of an increased number of follicles, resulting in access to multiple eggs. Inducing agents include, but are not limited to, gonadotropins and clomiphene citrate. Treatments preferably involve providing patients with recombinant human FSH (Gonal F, Serono Inc., Norwell, Mass., USA; Follistim, Organon Inc., West Orange, N.J., USA) after pituitary down-regulation with a gonadotropin-releasing hormone agonist (GnRHa; Lupron; TAP Pharmaceuticals Inc., Lake Forest, Ill., USA). Usually, follicular development is monitored by serial daily plasma estradiol measurement and vaginal ultrasound follicle examinations. Preferably, ovulation is triggered with about 10,000 IU intramuscular hCG (Profasi; Serono Ind) once at least two lead follicles measured about ≧18 mm with at least half the remaining follicles measuring about ≧15 mm, are detected.

The term “harvesting at least one oocyte” as used herein refers to procedures used to surgically retrieve eggs from an induced female. Eggs may be retrieved by any number of current methodologies common in the art including laparoscopy and, preferably, transvaginal ultrasound-guided oocyte aspiration. The latter is a technique which involves the introduction of a small needle through the vaginal wall under ultrasound guidance by a transvaginal ultrasonic probe. Preferably, oocytes are retrieved by ultrasound-guided transvaginal needle aspiration about 34-36 hours following hCG administration.

Techniques such as transvaginal ultrasound-guided oocyte aspiration also allow the clinician to obtain a sample of material representative of the cellular microenvironment in which the oocytes have developed.

The term “aspirating a matching follicular fluid” is defined herein to relate to fluid surrounding the oocyte that is taken up by the instrument used for retrieving the eggs from the female. The fluid sample may contain interstitial fluid, as well as other material from the ovary such as, but not limited to, extracellular matrix material or cellular material e.g., cumulous cells. Preferably, the fluid is set aside for subsequent analysis or analyzed in parallel with the oocyte it surrounded. It is the object of the invention to quantify certain molecular markers, i. e., determine follicular fluid parameters, within the follicular fluids that surround their matching oocytes, to determine which combination of molecular marker concentrations closely correlate with highly competent eggs.

The term “follicular fluid parameters” is a generic term for at least three different attributes for which the follicular fluid may be analyzed: androgen concentration, and concentration and balance of Th1 and Th2 cytokines.

The concentration of follicular fluid androgens relates to the measurement of male hormone levels in the follicular fluid. Preferably, the androgen is testosterone or a close structural derivative thereof. The levels of such compounds present in the follicular fluid samples matched to specific harvested oocytes are accomplished by conventional means.

Preferably, androgens such as: dehydroepiandrosterone, androstenedione, dihydrotestosterone, testosterone and/or androstenedione are determined by a conventional immunoassay. Dipsticks that could be adapted to semi-quantitatively measure follicular fluid androgen concentration are disclosed in U.S. Pat. No. 6,001,658, which is hereby incorporated by reference in its entirety. Generally, the assay is a radioimmunoassay as described by Costa et al., Braz J Med Biol Res, November 2004, Volume 37(11) 1747-1755, which is incorporated by reference in its entirety. Most preferably, the follicular fluid concentration of DHEA (dehydroepiandrosterone), DS, androstenedione (A), testosterone (T) and/or dihydrotestosterone (DHT) are determined. Preferably, follicular fluid samples are diluted to varying extents depending on the hormone to be measured. Thus, before the assay of each steroid, a follicular fluid sample is submitted to successive dilutions and the results obtained used to construct a curve that may be compared to a standard curve for the assay, indicating the ideal dilution for the steroid under study, which was the point closest to the ED₅₀ of the standard curve.

Preferably, the balance and/or level of expression of Th1 to Th2 cytokines in the follicular fluid sample is determined either by using conventional immunoassay techniques or RT-PCR. Th1 cells predominate in the non-pregnant state ovary and express interferon gamma, tumor necrosis factor (TNF) alpha, IL-2 and IL-3, the cytokines predominantly involved in cellular immunity, delayed hypersensitivity, tissue injury in infection and autoimmune disease. Th2 helper cells secrete IL-1, IL-4, IL-5, IL-6, IL-9, IL-10, and IL-13, cytokines that are involved in antibody production. The expression of the Th2 cytokines is postulated to down regulate the Th1 cytokines and vice versa. The balance between Th1 and Th2 cytokines largely predominates whether an induced shift towards Th2 during pregnancy establishes and perhaps co-ordinates a cytokine network that protects the developing oocytes or embryo from the maternal immune system. Clark, Am. J Reprod. Immunol, 1997: 38:75-78. The concentration of Th1 and Th2 cytokines is determined by any number of qualitative, quantitative and/or semi-quantitative methodologies common in the art. Preferably, such techniques rely on competitive or sandwich immunoassays. Most preferably, the ELISA methodology of Srivastava et al., Am J Reprod Immunol. September 1996;36(3):157-66 or Bili et al., J Assist Reprod Genet. February 1998;15(2):93-8, is employed. Dipsticks that could be adapted to semi-quantitatively measure follicular fluid Th1 and Th2 cytokine concentration are disclosed in U.S. Pat. No. 6,001,658, which is hereby incorporated by reference in its entirety. Alternatively, Th1 and Th2 cytokine expression levels may determined by assessing Th1 and Th2 cytokine mRNA isolated from cellular material in the follicular fluid sample, by RT-PCR in a procedure similar to the described by Kelemen et al., Am J Reprod Immunol. June 1998;39(6):351-5, which is hereby incorporated by reference.

The term “oocyte grade” is defined herein to relate to an assessment of morphological attributes or characteristics of an oocyte that are indicative of oocyte competency. Preferably, through a visible inspection of the harvested oocyte the skilled artisan can assign oocytes a grade of “1,” “2” or “3” by assessing a series of morphological traits. Specifically as used herein, there are three possible grades: Grade 1 embryos have substantially homogeneous cytoplasm, an intact polar body, normal oocyte shape, no visible cytoplasmic defects, no vitelline or zonae defects, as well as normal oolemma; Grade 2 oocytes have a visibly substantially homogenous cytoplasm and two of a) a fragmented polar body, b) abnormal oocyte shape, c) cytoplasmic droplets, d) vacuoles, e) a grainy spot, f) increased perivitelline space, and g) a darkened or defective zonae, and h) double oolemma. Finally, a grade 3 oocyte lacks visibly homogenous cytoplasm and has at least three of a) to g), above. Determining the presence or absence of each of these traits, preferably by light microscopy, is well within the skill of the ordinary artisan.

In oogenesis and paticularly meiosis I, a set of chromosomes, with two chromatids each, segregate to the “first polar body” or “PB-I” while the oocyte in MII retains the reciprocal chromosome complement. Preferably, the whole chromosome complement, i.e., the genome, of the PB-I is analyzed. Preferably such analysis is by comparative genomic hybridization.

Polar bodies are removed from their associated harvested oocytes by standard techniques known to those in the art. Genetic analysis of the polar body provides indirect information as to the genetic health of the oocytes with which it is associated. In female meiosis I, a set of chromosomes, with two chromatids each, segregate to the first polar body (PB-I) while the oocyte in metaphase II (MII) retains the reciprocal chromosome complement. Since the PB-I is thought to have no biological role once it has been extruded, the analysis of PB-Is allows the indirect characterization of the chromosome constitution of the MII oocyte. This means that if a segregation error occurs during this first meiotic division, and for instance, an extra chromosome is present in the MII oocyte, then the PB-I will show the complementary loss. Most embryo aneuploidies as well as most first trimester aneuploidies can be classified as originating in female meiosis I. However, FISH analysis results of first and second PBs has indicated that a sizable part of aneuploidy occurs in meiosis II, or at least, at the chromosome level, is expressed in meiosis II. Therefore, the detection of abnormal oocytes using genetic analysis may be performed in both, first and second PBs, but even biopsying on day 1, there is still enough time for genetic analysis results prior to transfer, and no cryopreservation is needed. Preferably, the differentiation of competent and incompetent oocytes is accomplished by using CGH analysis on the genetic complement of first PBs.

The terms “analyzing the genome” and “genetic analysis parameter” refer to the results of the genetic analysis of the first polar body.

A genetic analysis parameter may be determined by any technique known in the art for genomic genetic analysis. For example, Applied Biosystems' markets a system (Applied Biosystems Expression Array System®)based on chemiluminescence and that allows the skilled artisan to detect over 31,000 human genes using long, 60 bp DNA probes, which promote tight binding to target molecules. Alternatively, NimbleGen Systems Inc. markets a human whole-genome, long oligo microarray. NimbleGen's human array is composed of about 200,000 long oligo probes (60mers), with an average coverage of 5 probes per gene.

Preferably, the genetic analysis parameter is obtained by performing at one of least one of two tests: analysis of the short arm of chromosome 6 and comparative genomic hybridization.

Chromosome 6 is best known for the major histocompatibility complex (MHC), a region of 3.6 megabases (Mb) on band 6p21.3 of the short arm. The MHC has an essential role in the innate and adaptive immune system, and is characterized by high gene density, high polymorphism and high linkage disequilibrium. Mungall et al., Nature, 425, Oct. 23, 2003, pp 805-811. The invention envisages the detection of chromosomal abnormalities involving the short arm of chromosome 6 (6p). Preferably, chromosomal abnormalities are detected by using conventional cytogenetics and fluorescence in situ hybridization (FISH). Most preferably, chromosome-microdissection probes specific for 6p21 and 6p25, are used as described in Chen et al., Cancer Genet Cytogenet. August 2000;121(1):22-5. Alternatively, microsatellite markers on the short arm of chromosome 6 may be analyzed according to the methodology described in Van den Linden et al., Genes Immun. November 2001;2(7):373-80.

The term “comparative genomic hybridization analysis” or “CGH” relates to a molecular cytogenetic technique that allows the analysis of a full set of chromosomes in single cells. CGH, is a DNA-based method which does not involve cell fixation and allows for the analysis of the whole set of chromosomes and provides an accurate and reliable evaluation of aneuploidy (both hyperhaploidy and hypohaploidy).

Generally, CGH involves the use of polymerase chain reaction and a particular pair of relatively non-specific, i.e., degenerate, PCR-primers to amplify in parallel, stretches of DNA along all chromosomes present in a test and reference whole genome template sample. Preferably, the test chromosomes are from a first polar body (PB-I) and the reference chromosome are derived from any euploid human haploid cell. Next, the amplified DNA product of the test reaction is labeled with one visually detectable label, whereas the amplified DNA product of the reference reaction is labeled with another visually detectable label, thus providing two alternate probes. Preferably, the two different labels are fluorophores that fluoresce at different wave lengths. The test and reference probes are then hybridized to a spread of metaphase chromosomes derived from healthy human cells. Preferably, a ratio of the intensity of the labeled test and reference probes hybridized to the metaphase chromosomes will provide information whether the test genomic sample contains more or less DNA than the reference sample. Ideally, when amplifying a healthy normal, i.e., euploid, test genomic sample, the ratio of labeled test and reference probes hybridized to the metaphase chromosomes is about 1:1. Any deviation from that ratio, i.e., the genetic analysis parameter, indicates that the test genomic sample contains more or less DNA present than the healthy reference sample, thereby indicating aneuploidy. This ratio is referred to interchangeably herein as the “comparative genomic hybridization parameter.” However, one of skill in the art will know that a perfect 1:1 ratio is usually not possible given technical experimental variations. As such, a comparative genomic hybridization parameter indicative of a euploid oocyte or blastomere is from about 0.8:1 to about 1.2:1, preferably about 0.9:1 to about 1.1:1 and most preferably 1:1. Preferably the determination of whether a comparative genomic hybridization parameter is indicative of a euploid oocyte or blastomere is made using a computer software such as SmartCapture™ software and Vysis Quips™ CGH software, both supplied by Vysis, for example.

Fertilizing the oocytes to obtain an embryo is performed by standard in vitro fertilization techniques. Preferably, isolated oocytes are fertilized by intracytoplasmic sperm injection (ICSI). Most preferably, in establishing a database envisaged by the invention, a plurality of oocytes are fertilized with the same sperm from the same donor. Using the same sperm minimizes non-oocyte variability in the standardized evaluation of embryonic development using the graduated embryonic scale.

Prior to the first cell-division, the freshly fertilized pre-embryo displays two pronuclei, one derived from each parent, and which contain the genetic material. From 24 to 30 hours after fertilization (day 1), the embryo should have divided into 2 cells, by day 2 it should have 4 cells, and by day 3 there should be 7 to 8 cells. Until day 3, all the cells are identical. Embryonic development is controlled by maternal genes in the egg until around the 8-cell stage, when the potential for further development comes under the control of the embryo itself. By day 5 the cells have started to differentiate into specific types, each with a specialized function. The outer cells will eventually form the trophectoderm (placenta and fetal membranes). Secretions from inner cells collect in a central cavity, called the blastocele, and become the amniotic fluid. Specialized cells on the inner surface of the morula form the inner cell mass (ICM) that eventually develops into the fetus. This complex creation is now called a blastocyst. As the cavity fills with fluid, the blastocyst expands and eventually “hatches” from the zona pellucida. The hatched blastocyst then implants into the endometrium 6 to 7 days after ovulation.

The term “embryo grade” is referred to herein to be a score based on an evaluation of embryos were by graduated embryo score (GES) on days 1, 2, and 3 of culture. With GES, each embryo is separately examined through a series of microscopic assessments throughout a period of 72 hours following egg insemination. The maximum allotted GES score is 100. A four-year evaluation of embryos derived from the eggs of thousands of women under 40 has revealed that embryos with a GES score of about 70-100 each have better than a 35% likelihood of implanting successfully as compared to less than 20% when the GES score is below about 70. Embryo implantation potential decreases rapidly, progressively, and proportionately to well below 10% per embryo by the time the egg provider reaches 43 years of age. The GES system and its derivation have been previously described in detail in Fisch et al., Hum. Reprod. 2001; 16:1970-5, which is hereby incorporated by reference. Briefly, (see Table 1) GES is the sum of three, weighted, interval evaluations of early developmental milestones, totaling a possible 100 points. Embryos are first evaluated at 16-18 hours after insemination for the presence of nucleolar alignment along the pronuclear axis. Nucleolar alignment was found to be important and was given increased significance in the GES scoring system. An embryo with nucleoli aligned along the pronuclear axis is given 20 points. A second evaluation occurs at about 25-27 hours after insemination for the presence of regular and symmetrical cleavage, and if so, for percentage of fragmentation. If fragmentation is absent 30 points are assigned. If there is less than 20% fragmentation, 25 points are given. However, if fragmentation is greater then 20% then no points are given. Early and regular cleavage is noted to be especially important and is given the highest weight. A final evaluation of morphologic characteristics (cell number and fragmentation) occurs 64-67 hours after insemination (day 3 of culture). If an embryo is not cleaved at 25-27 hours, but develops into a grade A embryo (≧7 cells, <20% fragmentation) on day 3, points for fragmentation are awarded retrospectively.

TABLE 1
Graduated Embryonic Score (GES)

| | Hours after | | |
|---|---|---|---|
| Evaluation | insemination | Developmental Milestone | Score |
| 1 | 16-18 | Nucleoli aligned along | 20 |
| | | pronuclear access | |
| 2 | 25-27 | Cleavage regular and symmetrical | |
| | | Fragmentation^{a} | |
| | | Absent | 30 |
| | | >20% | 25 |
| | | <20% | 0 |
| 3 | 64-67 | Cell Number and grade^{b}: | |
| | | 7-cell I; 8-cell I; 8-cell II; | 20 |
| | | 9-cell I | |
| | | 7-cell II, 9-cell II, 10-cell I; | 10 |
| | | 11-cell I Compacting I | |
| | | Total score | 100 |

^{a}If the embryo was not cleaved at 25-27 hours, grading of fragmentation should occur at the 64-67 hour evaluation if the embryo reached the seven cell stage and had <20% fragmentation.
^{b}Grade I = symmetrical blastomeres and absent fragmentation. Grade II = slightly uneven blastomeres and <20% fragmentation. Grade III = uneven blastomeres and >20% fragmentation. Grade A embryos are seven or more cells with <20% fragmentation

Additionally, the invention envisages performing a CGH procedure on blastomere biopsies from about 3 to about 6 day old embryos in culture. “Blastomere biopsy” is a technique that is performed by removal of one or two cells (blastomeres) from the 6 to 8 cell pre-embryo stage for the purpose of preimplantation analysis. On the third day following fertilization, the embryo is at the cleavage stage, and a cell may be carefully removed for genetic analysis. With the embryo maintained in position by gentle suction of the holding pipette, an opening in the outer shell called the zona pellicuda is made using a micro needle. A new micropipette is the used to remove a cell by means of aspiration. At this early stage of embryo development, all of the cells have the same potential for development, therefore, removal of a cell from the embryo is not detrimental and the embryo should continue to develop normally following the procedure. The genetic complements of the cells that have been removed are then tested. Preferably they are tested by comparative genomic hybridization.

Delivering or transferring an embryo to a female to facilitate embryonic development generally involves insertion of a developing embryo into the female uterus. Methods of transferring embryos are well known in the art. Preferably, the procedure entails gentle placement of embryo(s) within 1-2 cm of the roof of the uterine cavity under direct ultrasound visualization. Preferably, embryos are transferred atraumatically using a soft Teflon catheter e.g., a Wallace catheter (Cooper Surgical, Shelton, Conn.), under ultrasound guidance.

One of skill in the art will understand that a “database” as used herein involves the storage and statistical analysis of the relative importance of various factors to the successful development of a fetus and baby. Specifically the database will preferably, correlate the development the embryo/fetus/baby with at least one follicular fluid parameter, the oocyte grade of the embryo, at least one genetic analysis parameter; and the embryo grade. Most preferably, the database will provide information on the relative importance of the concentrations of various components found in the follicular fluid in determining the competency of an oocytes with which it is matched. Such information will allow the clinician to readily and accurately predict the competency of an oocyte immediately following its retrieval by routine analysis of matching follicular fluid.

The term “freezing” an oocyte refers to the use of standardize cryopreservation techniques of freezing oocytes. The term “thawing” an oocyte refers to the use of standardized techniques of thawing oocytes.

The ability to differentiate a competent oocyte from an incompetent oocyte would allow optimal egg selection aimed at inter alia: a) improving the outcome of human IVF/ET in terms of increased success rates and the minimization of multiple pregnancies; b) establishing a sound basis for egg selection in order to improve the ability to successfully cryopreserve and bank human eggs for fertility preservation; c) establishing a basis for simplifying and propagating ovum donation services through establishing egg banking and exportation of cryopreserved oocytes; and d) establishing a pool of data that can be retrospectively used to correlate the environmental, medical and/or nutritional factors to which women are exposed, with the likelihood of producing competent oocytes.

Given current technology, it is very difficult both financially and technically to determine whether a normal-looking, day-2 or 3, post-ICSI embryo or a day 5-6 post-ICSI blastocyst is in fact competent or not. In order to confirm embryos competency in the past, clinicians needed to genetically analyze embryos using the potentially disruptive, time consuming and expensive blastomere biopsy technique. The inventors have discovered that embryo aneuploidy does not occur spontaneously, but rather it is directly traceable to oocyte aneuploidy. The methods disclosed herein circumvent the necessity of blastomere biopsies by focusing instead on analysis of the oocyte PB-I and/or the follicular fluid aspirated with an oocyte.

The inventors have discovered inter alia, that when the chromosome complement of a first polar body (PB-D) is normal, it alone is in more than 80% of cases sufficient to predict that an embryo which has developed from an oocyte associated with the PB-I is also normal and competent. In other words the presence a normal chromosome complement of a first polar body (PB-I) is sufficient to predict that the oocyte with which it was associated, assuming successful fertilization and subsequent embryogenesis to the embryo/blastocyst stage, strongly suggests that the embryo/blastocyst will also be competent. If a competent embryo/blastocyst is transferred to a female is capable of carrying an embryo to term and there is no implantation dysfunction, such a competent embryo will almost always produce a viable euploid pregnancy that will result in the birth of a genetically healthy, i.e., euploid baby. Accordingly, the clinician is able to determine the competence of a harvested oocyte based on an analysis of the genetic complement of its associated PB-I.

Specifically, the inventors have found that when a euploid oocyte (as determined by PB-I analysis) fertilized with competent sperm develops into an embryo; that embryo will almost always be euploid. Example 6. Furthermore, the inventors have discovered that embryo euploidy is not only necessary for embryo competency but also sufficient to predict it. Example 7. Taken together, a if a euploid oocyte fertilized with competent sperm develops into an embryo; the resulting embryo will almost always be competent.

As such, the inventors have determined that embryo incompetence is almost always due to oocyte aneuploidy (occurring during meiosis and not post-fertilization) and that if a fertilized euploid oocyte develops into an embryo; it is almost always euploid and competent, as well. Given the techniques disclosed herein, the clinician can be substantially certain of this conclusion without having to perform a potentially destructive blastomere biopsy on a candidate embryo.

Most preferably, the methods disclosed herein allow the clinician to harvest a large number of oocytes, fertilize them and wait to see how many embryos develop. Of those embryos, the clinician can then identify euploid and competent embryos for transfer. Euploid and competent embryos are identified by the fact that the PB-I associated with the oocyte from which the embryo derived, were genetically normal. Most preferably, the genetic analysis of a PB-I is conducted by comparative genomic hybridization. These methods allow the clinician to transfer only those embryos derived from euploid oocytes. As such, the clinician can transfer a minimum number of euploid and competent embryos without having to perform a blastomere biopsy and with the knowledge that multiple pregnancies can be mitigated.

Oocyte competency also depends on the characteristics of the cellular microenvironment in which the oocyte develops. Preferably, those characteristics are determined through an analysis of the follicular fluid aspirated during retrieval of an oocyte from a female. The inventors obtained samples of follicular fluid surrounding each of the oocytes harvested for the study described in Examples 6 and 7. These are referred to as matching follicular fluid samples. Given the fact that the inventors discovered that the presence a euploid first polar body (PB-I) is sufficient to predict the ability of its associated oocyte to yield a euploid and competent embryo; the inventors also envisage being able to retrospectively correlate the levels of follicular fluid constituents with embryo competency.

Accordingly, the clinician is able to determine the euploidy of a harvested oocyte based on an analysis of the genetic complement of its associated PB-I and/or will be able to determine the competency of a harvested oocyte based on an analysis of its matching follicular fluid to determine if the biochemical constituents therein correlate with oocyte and embryo competency.

Preferably, the clinician analyzes the follicular fluid aspirated with the harvested oocyte and determines whether the biochemical make up of the follicular fluid correlates with oocyte competency. Preferably, the method envisages analyzing the concentration of androgens and/or the balance of the concentration of Th1 and Th2 cytokines, i.e., follicular fluid parameters, in a follicular fluid sample and selecting those oocytes whose matching follicular fluid samples have follicular fluid parameters that are indicative of oocyte competency.

The invention enables identification of at least one follicular fluid parameter that will allow for the differentiation between “competent oocytes,” i.e., those that following in vitro fertilization, development to the blastocyst stage and subsequent embryo transfer (ET) are most likely to result in a healthy and normal conceptus and those that are not. In one embodiment of this aspect of the invention, the inventors will correlate at least one follicular fluid parameter within the follicular fluid that surround their matching oocytes, with oocyte grade, at least one oocyte genetic analysis parameter, a graded embryo score of a resulting embryo, a healthy pregnancy and delivery. The analysis of these data will allow for the determination of certain follicular fluid parameter values as indicators of oocyte and embryo competency.

The creation of a database correlating various follicular fluid parameters with oocyte and embryonic competency as described herein, enables the clinician to analyze the follicular fluid aspirated with future donated oocytes to determine the matching oocyte's competency. Accordingly, it is envisaged that a clinician obtains a number of donated oocytes, e.g., by the methods described herein, and analyzes their matching follicular fluid's parameters. Based on correlations in the database described herein, the follicular fluid parameters will allow the clinician to predict which donor oocyte is competent and, therefore, which to embryos to transfer.

The invention also envisages making an egg bank by freezing and storing oocytes whose PB-Is were determined by the methods of the invention to be euploid. The invention further envisages freezing, storing, thawing and transferring euploid embryos.

The inventors believe that central to the development of a successful and practicable oocyte cryopreservation technique is to gauge success based upon the quality of the oocytes being frozen rather than the technique used to freeze them. Since most of the oocytes currently being frozen are derived from women in their late 30's who find themselves in a race with their biological clock, it is likely that the vast majority of oocytes frozen in the past, were aneuploid and thus incompetent thereby explaining the unsatisfactory results hitherto reported. Therefore, the invention envisages determining the aneuploidy of a harvested oocyte based on an analysis of the genetic complement of its associated PB-I and/or the competency based on an analysis of its matching follicular fluid; and only cryopreserving those oocytes that are deemed euploid; or euploid and competent.

Given the physiological rigors of freezing and thawing an oocyte, it can be assumed that a portion of frozen euploid oocytes will not survive in a condition to be able to develop into an embryo following fertilization. However, those that do, become euploid embryos upon fertilization with euploid sperm.

Therefore, another aspect of the invention relates to a method of selecting an oocyte for cryopreservation by selecting only euploid; or euploid and competent oocytes for the procedure. In one embodiment, a clinician determines the competency of the oocyte by analyzing the follicular fluid parameters of its matching follicular fluid. In another embodiment, the clinician determines the ploidy of the oocyte by analyzing the genetic complement of their associated PB-I. In the preferred embodiment, the genetic analysis of a PB-I is conducted by comparative genomic hybridization.

In another embodiment, only oocytes that have been determined to be euploid are fertilized and allowed to develop to the blastocyst stage at which time they are frozen. In another embodiment, an egg bank is created by a female human patient wherein over time competent eggs are identified and frozen. In a further embodiment, an egg bank is created from a collection of euploid frozen oocytes donated by a variety of females.

In the past the efficacy and safety of potential fertility drugs could only be ascertained indirectly by cumbersome, inaccurate surrogate physiological parameters. Therefore, another aspect of the invention relates to methods of identifying compounds that are useful as fertility drugs. In one embodiment, the efficacy and safety of potential fertility drugs may be assessed directly and prospectively by PB-I genomics and follicular fluid parameters. For example, the success of a course of treatment of a candidate fertility drug could be measured by the percentage euploid oocytes produced. Using the methods described herein, oocyte polidy is measured by analyzing one or more follicular fluid parameters or the genetic complement of a PB-I.

Most preferably, because the inventors discovered that when a euploid oocyte fertilized with competent sperm develops into an embryo—the resulting embryo will almost always be competent; it follows that an increase in oocyte euploidy as a result of a fertility drug treatment over time also correlates with its effectiveness. In other words, an increase in the production of euploid oocytes correlates with the increased fertility of a female.

One embodiment of this aspect of the invention relates to animal models designed to assess the safety of pharmaceutical agent compounds that are being developed for human use in a variety of therapeutic areas. For example, female animals are given a course of treatment of a candidate compound. Preferably, during or after the course of treatment, oocytes are harvested and analyzed for euploidy by way of genetic analysis of their PB-Is and/or competency analysis of their matching follicular fluid samples. If the percentage of competent and/or euploid oocytes increases over time in response to the course of treatment, then the compound is a promising fertility drug. In the preferred embodiment, genetic analysis is by CGH.

The invention further envisages the use of the methods disclosed herein for new drug development and for quality assurance testing for drug safety and test kits tests as diagnostic products.

### EXAMPLE 1

Ovarian Stimulation:

Patients undergo ovarian stimulation using similar protocols at all sites. All patients receive Lupron (TAP, Pharmaceuticals) in a long protocol after pretreatment with oral contraceptive pills for 1 to 3 weeks. Ovarian follicular development is stimulated with rhFSH at doses of 225-450 IU a day. Ovulation is triggered when at least 2 follicles are 18 mm and half the remainder is ≧15 mm. Oocytes are recovered transvaginally under ultrasound guidance 34.5 hours later. All monitoring of controlled ovarian hyperstimulation (COH) as well as ER's and ET's is performed by one of five physicians.

### EXAMPLE 2

A. Oocyte and Polar Body Recovery

Only mature oocytes, i.e., those that are considered to be at the metaphase II stage (having extruded the PB-D) are used. The zona pellucida is removed using acid Tyrode's. After that, MII-oocytes and their PB-Is are isolated and washed in three PBS/0.1% polyvinyl alcohol (PVA) droplets. The single cells are transferred to individual PCR tubes and the presence of the single cell inside the tube is ascertained. Finally, 1 ml of sodium dodecyl sulphate (SDS, 17 mM) and 2 ml of proteinase K (125 mg/ml) are added and the sample is overlaid with light mineral oil. The lysis is performed by incubating at 37° C. for 1 h followed by 10 min at 95° C. to inactivate proteinase K.

B. Whole Genome Amplification

Single cell DNA is amplified using degenerate oligonucleotide primed PCR (DOP-PCR) as previously described (Wells et al., 2002) with some modifications. In brief, each PCR tube contained 1× buffer, 2 mM DOP primer (CCGACTCGAGNNNNNNATGTGG; SEQ ID NO: 1), 0.2 mM dNTPs and 2.5 U of SuperTaq Plus polymerase (Ambion, Austin, Tex.) in a final volume of 50 ml. The sample is spun and heated to 94° C. for 4.5 min; 8 cycles of 95° C. for 30 s, 30° C. for 1.5 min and 72° C. for 3 min; 40 cycles of 95° C. for 30 sec, 56° C. for 1 min and 72° C. for 3 min with a final extension step of 72° C. for 8 min. The PCR program is carried out in a T gradient thermocycler 2119 (Biometra, Goettingen, Germany) or alternatively in a 9700 PE thermocycler (Applied Biosystems, Norwalk, USA). Stringent precautions against contamination are taken. Negative controls are included in each experiment to test the reaction solutions and the phosphate-buffered saline used for washing the single cells in the isolation step. The negative controls are subjected to the entire procedure. No DNA and no hybridization signal should be present after the DOP-PCR and the CGH experiment, respectively. Genomic DNA extracted from peripheral blood diluted to 100 pg/ml or isolated and lysed single buccal cells, both from a normal female were also amplified and used as a reference sample in the CGH experiment.

C. Nick Translation and Probe Preparation

Whole-genome amplification products are fluorescently labeled by Nick Translation (Vysis, Downers-Grove, USA) according to the manufacturer's instructions. PB-I DNAs (test) are labeled with Spectrum Red-dUTP (Vysis), whereas reference DNA is labeled with Spectrum Green-dUTP (Vysis). The reaction time is adjusted to obtain a probe of a suitable size, and assessed by electrophoresis of 9 ml of product in a 2% agarose gel. Labeled reference and test DNA are mixed and ethanol precipitated with 10 mg of Cot-1-DNA. The pellet is dried and redissolved in 10 ml of hybridization mixture (50% formamide, 2×SSC, 10% dextran sulphate, pH 7). Comparative genomic hybridization Normal male (46, XY) metaphase spreads (Vysis) are dehydrated through an alcohol series (70%, 85%, and 100% for 2 min each) and air dried. The slides are then denatured in 70% formamide, 2×SSC at 73° C. for 5 min and taken through a cold alcohol series and air dried. The probes are denatured at 73° C. for 10 min and applied to the slide; a coverslip is placed on top and sealed with rubber cement. Hybridization is performed in a moist chamber at 37° C. for 36-72 h to evaluate the minimal hybridization time to ensure reliable results. After hybridization, the slides are washed at high stringency in 0.4×SSC/0.3% NP-40 at 73° C. for 2 min, 2×SSC/0.1% NP-40 for 2 min and dipped in distilled water before being dehydrated through an alcohol series and air dried. Finally, the slides are mounted in Vectashield (Vector Labs, Peterborough, UK) containing DAPI to counterstain the chromosomes and nuclei.

D. Microscopy and Image Analysis

Metaphase preparations are examined using an Olympus BX 60 epifluorescence microscope equipped with a high-sensitivity camera and filters for the fluorochromes used. An average of 10 metaphases per hybridization are usually captured and analyzed using SmartCapture™ software and Vysis Quips™ CGH software, both supplied by Vysis. The average red/green fluorescent ratio for each chromosome is determined by the CGH software. In regions where the DNA sequence copy number of the test is identical to the reference DNA, the CGH profile shows no fluctuation and the ratio is expected to be close to 1.0. Deviations of the ratio below 0.8 (the test DNA is under-represented) or above 1.2 (the test DNA is over-represented) are scored as loss or gain of material in the test sample, respectively. Deviations of the ratio but within the threshold cut-off of 0.8 or 1.2 are also annotated to evaluate the sensitivity of the technique.

### EXAMPLE 3

Embryo Culture

Metaphase II (MII) oocytes are fertilized using ICSI 4-6 hours after retrieval in all cases. Embryos are cultured individually in 50 μl droplets of PI (Irvine Scientific) under oil at 37° C. in a 6% CO2, 5% 02, 89% N2 environment. All embryos are microscopically evaluated serially over a period of 72 hours following ICSI using the GES system placing special emphasis on cell number and percentage of fragmentation. All embryos are transferred to blastocyst medium 46 hour post-ICSI.

### EXAMPLE 4

A. Oocyte Cryopreservation

After egg collection, maintain all eggs in culture for 3 hours before attempting cryopreservation. Strip all eggs in Hyaluronidase and select matured (MID) eggs under stereo microscope. Place matured eggs (MII) to be frozen into warm modified HTF and then place them on to the bench at room temperature for 10 min to cool down (approx. 22° C.). Expose to 7.5% ethylene glycol and 7.5% DMSO in modified TCM 199 with 20% SSS (synthetic serum supplement) for 8 min. Place into 20% ethylene glycol and 20% DMSO+0.2M+0.1M Ficoll sucrose for a further 90 sec. Then oocytes will be loaded onto the cryoloop for 90 seconds. After 90 sec cryoloop will be plunged into liquid nitrogen immediately.

B. Thawing the Cryopreserved Oocytes

Place cryoloop at 37° C. 0.3 M sucrose in modified TCM 199 with 20% SSS for 2 min, then place in 0.15M sucrose for 3 min, and they will be kept in modified TCM 199 with 20% SSS for another 5 min. Then wash eggs through 4-5 drops modified HTF, 6-8 drops of plain HTF+10% SSS, and place them in the incubator. Undertake ICSI on all mature thawed eggs only after 2-3 hours in culture, after which any cytoskeletal damage that may have occurred during freezing will have had an opportunity to repair itself.

### EXAMPLE 5

Blastomere Biopsy

Blastomere biopsy may be carried out in HEPES-buffered medium overlaid with pre-equilibrated mineral according to Magli et al., Human Reproduction, Vol. 14, No. 3, 770-773, March 1999. Zona pellucida is chemically breached (acidic Tyrode's solution at pH 2.35) and the selected blastomere gently are removed. If fragments are present in the perivitelline space, they are also removed during the procedure. The nucleus is fixed on a glass slide (methanol-acetic acid 3:1), dehydrated in rising ethanol dilutions (70%, 85% and 100%) and incubated with the hybridization solution at 37° C. in a humidified chamber, for 4 h.

### EXAMPLE 6

This experiment set out to evaluate egg and embryo ploidy by performing genetic testing on DNA derived from PBI-Is as a predictor of the associated egg's ability to subsequently spawn euploid embryos (as determined by CGH on the PB-II and a blastomere of the post-fertilized egg). The inventors discovered that a euploid egg was an accurate predictor of subsequent embryo eupoloidy. The ability to make this diagnosis on an egg provides the clinician with methods for selecting one embryo for transfer with the expectation of a high probability of a normal viable pregnancy regardless of the age of the woman who produced the egg.

The experiment involved the stimulating the ovaries of 14 hand picked egg donors (mean age=26±3.3 years). A total of 130 mature eggs were harvested and their polar bodies (PB-D) were removed. The PB-Is were then analyzed using CGH. Next each mature egg was fertilized by intracytoplasmic sperm injection (ICSI) using donor sperm derived from a licenced sperm bank. Following fertilization, the second polar body (PB-II) was extracted and processed for CGH.

All embryos were maintained in culture for 72 hours post ICSI and then graded according to the GES system. A blastomere bioposy was performed on each embryo by removing a single embryonic cell (blastomere) from all GES=>70/100 scoring embryo. The DNA from each blsastomere was tested by CGH. All embryos were cultured for an additional 24-48 hours in specialized culture media. Those embryps that those embryos that developed into blastocysts were cryopreserving and stored. The blastocysts derived from pre-fertilized eggs with a normal karyotype (euploid) and which upon fertilization spawned euploid embryos that had exhibited karyotypically normal PB-II's and blastomeres were deemed to be most likely to be “competent” i.e., capable of producing a normal viable pregnancy.

As indicated above, the karyotypic lineage of each embryo was tracked using CGH performed once in the pre-fertilization stage (PB-I) and twice post-fertilization (PB-II followed by blastomere), showed that a normal karyotype of the pre-fertilized egg was predictive of a subsequent normal karyotypic lineage in of the post-fertilized egg.

The results of this experiment a shown in Table 2 and demonstrate that:

1) Greater than 90% of harvested oocytes were karyotyped by CGH of associated PB-I's.

2) All of the 130 oocytes were fertilized but only 58 (45%) developed to the blastocyst stage.

3) Of the 58 blastocysts, approximately 55% were aneuploid as determined by CGH. Of the 58 embryos only 25 (43%) were euploid as determined by CGH.

4) Most importantly when looking back at the oocytes that gave rise to the 25 euploid embryos, it was observed that wherever a euploid oocyte (as determined by PB-I CGH) was fertilized by ICSI and developed to an embryo, the corresponding embryos (and blastocysts) were likewise euploid in all but one case studied. In that case, (“Holli 23”; case 28), the aneuploidy observed in the embryo derived from a euploid oocyte may have been the result of a defective sperm, embryo mosaicism or an embryonic lethal mutation that is not detecable by CGH. See Table 2 below:

| | Case | Sample | | | | | |
|---|---|---|---|---|---|---|---|
| Name | # | # | PB 1 | PB 2 | BB | Gender | Result |
| Jazmin 14 | 1 | 2 | ABN | NL | ABN | XY | ABN |
| Jazmin 14 | 2 | 7 | NL | NL | NL | XY | NL |
| Jazmin 14 | 3 | 5 | ABN | ABN | ABN | — | ABN |
| Wendy 4 | 4 | 6 | ABN | ABN | ABN | XX | ABN |
| Wendy 4 | 5 | 15 | NL | NL | NL | XX | NL |
| Wendy 4 | 6 | 17 | NL | NL | NL | XX | NL |
| Wendy 4 | 7 | 21 | ABN | ABN | ABN | XY | ABN |
| Theresa 15 | 8 | 2 | NL | NL | NL | XY | NL |
| Theresa 15 | 9 | 3 | ABN | ABN | NL | XY | ABN |
| Theresa 15 | 10 | 4 | ABN | ABN | ABN | XY | ABN |
| Theresa 15 | 11 | 5 | ABN | ABN | ABN | XY | ABN |
| Theresa 15 | 12 | 6 | ABN | ABN | ABN | XY | ABN |
| Theresa 15 | 13 | 8 | NL | N/A | NL | XY | NL |
| | | | | Freg | | | |
| | | | | PB | | | |
| Theresa 15 | 14 | 9 | ABN | ABN | ABN | XY | ABN |
| Theresa 15 | 15 | 11 | NL | NL | NL | XY | NL |
| Theresa 15 | 16 | 17 | NL | NL | NL | XY | NL |
| Theresa 15 | 17 | 18 | NL | NL | NL | XX | NL |
| Theresa 15 | 18 | 20 | ABN | ABN | ABN | XY | ABN |
| Theresa 15 | 19 | 24 | ABN | ABN | ABN | XY | ABN |
| Theresa 15 | 20 | 26 | NL | NL | NL | XY | NL |
| Theresa 15 | 21 | 29 | ABN | ABN | ABN | XY | ABN |
| Jeannie 13 | 22 | 4 | ABN | ABN | ABN | XX | ABN |
| Jeannie 13 | 23 | 5 | ABN | ABN | NL | XX | ABN |
| Jeannie 13 | 24 | 7 | ABN | ABN | ABN | XX | ABN |
| Jeannie 13 | 25 | 17 | NL | NL | NL | XX | NL |
| Holy 23 | 26 | 6 | NL | NL | NL | XY | NL |
| Holli 23 | 27 | 11 | NL | NL | NL | XY | NL |
| Holli 23 | 28 | 12 | NL | ABN | ABN | XY | ABN |
| Holli 23 | 29 | 13 | NL | NL | NL | XX | NL |
| Holli 23 | 30 | 14 | NL | NL | NL | XX | NL |
| Rachel 7 | 31 | 4 | NL | NL | NL | XX | NL |
| Rachel 7 | 32 | 8 | ABN | ABN | ABN | XY | ABN |
| Rachel 7 | 33 | 10 | ABN | ABN | ABN | XX | ABN |
| Tania 2 | 34 | 1 | ABN | ABN | ABN | XX | ABN |
| Tania 2 | 35 | 3 | NL | NL | NL | XY | NL |
| Theresa 15 | 36 | 1 | NL | NL | NL | XY | NL |
| Theresa 15 | 37 | 4 | ABN | ABN | ABN | XX | ABN |
| Theresa 15 | 38 | 5 | ABN | ABN | ABN | XX | ABN |
| Rachel 40 | 39 | 1 | ABN | ABN | ABN | XY | ABN |
| Rachel 40 | 40 | 2 | NL | N/A | NL | XY | NL |
| | | | | Freg | | | |
| | | | | PB | | | |
| Rachel 40 | 41 | 3 | ABN | ABN | ABN | XY | ABN |
| Rachel 40 | 42 | 6 | ABN | NL | ABN | XY | ABN |
| Rachel 40 | 43 | 7 | NL | NL | NL | XY | NL |
| Rachel 40 | 44 | 9 | NL | NL | NL | XY | NL |
| Rachel 40 | 45 | 11 | NL | NL | NL | XX | NL |
| Rachel 40 | 46 | 12 | ABN | N/A | ABN | XX | ABN |
| | | | | Freg | | | |
| | | | | PB | | | |
| Rachel 40 | 47 | 13 | NL | NL | NL | XX | NL |
| Rachel 40 | 48 | 14 | ABN | N/A | ABN | XY | ABN |
| | | | | Freg | | | |
| | | | | PB | | | |
| Rachel 40 | 49 | 17 | ABN | N/A | ABN | XX | ABN |
| | | | | Freg | | | |
| | | | | PB | | | |
| Stephanie | 50 | 3 | N/A | ABN | ABN | XX | ABN |
| 22 | | | | | | | |
| Stephanie | 51 | 4 | ABN | ABN | ABN | XX | ABN |
| 22 | | | | | | | |
| Laureen | 52 | 3 | NL | NL | | | NL |
| Laureen | 54 | 5 | ABN | ABN | | | ABN |
| Laureen | 55 | 7 | ABN | ABN | | | ABN |
| Laureen | 56 | 10 | NL | NL | | | NL |
| Laureen | 57 | 13 | ABN | ABN | | | ABN |
| Laureen | 58 | 15 | NL | NL | | | NL |

NL: Normal
ABN: Abnormal
N/A: Not amplified
Freg: Fragmented
PB: Polar Body
BB: Blastomere biopsy

The data in Table 2 represent cases where fertilization of oocytes resulted in the development of blastocysts five to six days post-ICSI. There were 23 cases, of euploid oocytes (PB-I). In 22 of these cases (22/23=96%), the corresponding zygotes (PB-II) as well as the 72-hour embryos (BB) were likewise euploid. In only one (1) case (“Holli 23”; Case 28) the corresponding zygote and embryo were both aneuploid (BB (suggesting mosaicism in the embryo). There were 25 cases, of aneuploid oocytes. In 23 of these cases (23/25=92%), the corresponding zygotes as well as the 72-hour embryos were both aneuploid. In two (2) cases, the zygotes were aneuploid (PB-II) while the embryos were both euploid (BB, suggesting a technical error).

Therefore, in the vast majority of these cases, a clear correlation exists between the karyotype (ploidy) of the oocyte of origin (PB-I) and the subsequent ploidies of both the zygotes (PB-II) and embryos (BB). In other words, when the oocyte was euploid, so were the resulting post-fertilization karyotypes of the corresponding zygotes and embryos in the vast majority of cases (96%). Conversely, when the oocyte of origin was aneuploid, so were the corresponding zygotes and embryos in virtually all cases.

This study confirms that PB-I euploidy provides a high level of confidence (29/32; 91%) that the corresponding post-ICSI embryo(s) will likewise be euploid. As such, this methodology represents an a rational basis excellent method for assessing oocyte/embryo competence and establishes a rational basis for performing PB-I biopsies on all M-II (i.e. mature) oocytes to select a single competent embryo/blastocyst for transfer to a receptive uterus with a high expectation of initiating a viable pregnancy and an euploid, fetus and baby.

### EXAMPLE 7

Fresh blastocysts were derived from 8 egg providers, who were less than 42 years of age. In 3 of the cases the oocytes were obtained from donors and in the remaining 5 cases the egg provider underwent embryo transfer to her own uterus. The egg providers all underwent ovarian stimulation with gonadotropins. Thirty five hours following hCG transvaginal oocyte retrievals were performed. In each case, up to 10 M-II oocytes were arbitrarily selected for PB-I biopsy and CGH. Up to 2 (average 1.3 per recipient), euploid competent blastocysts, as determined by PB-I analysis of the oocytes from which they derived, were transferred to the uterus of each recipient. Seven of the eight recipients (7/8=87.5%) achieved positive blood pregnancy tests and all seven developed ultrasound confirmed clinical pregnancies. These results in conjunction with those of Example 6 indicate that it is possible, to select and transfer a single competent euploid embryo/blastocyst (as determined by PB-I analysis of the oocytes from which they derived) with the expectation that this will result in a viable pregnancy.

### EXAMPLE 8

This study is similar in design to Examples 6 and 7 with the difference that up to 10 oocytes will be cryopreserved for a period of 7 days following initial PB-I genomic testing by CGH. In some or all cases the oocytes will be divided between two separate recipient couples. Initially, PB-I biopsies will be performed on all MII oocytes. The PB-I biopsies derived from the (up to) 10 selected oocytes will be evaluated immediately using CGH. The amplified DNA derived from the polar bodies biopsied from remaining oocytes will be frozen for subsequent retrospective analysis at the discretion of the principal investigators. Those oocytes that are not vitrified will undergo ICSI within 4-6 hours of oocyte collection and will be taken to the blastocyst stage (day 5-6 post-ICSI) and vitrified for subsequent dispensation. Upon thawing, all previously vitrified oocytes will undergo ICSI using the sperm derived from the partners of embryo recipients (or from accredited, designated sperm donors). Forty-six hours later, early embryo will be graded conventionally as well as by using Graduated Embryo Scoring (GES). Five (5) to 6 days post-ICSI up to 2 blastocysts derived from previously vitrified oocytes that were determined by CGH to be euploid, will preferentially be transferred to the uteri of each designated embryo recipient. In the event that less than two (2) such blastocysts are available, an attempt will be made to make up such a shortfall with morphologically normal looking blastocysts derived from those oocytes that had not been vitrified. Since the karyotype of the oocytes of origin of these supernumerary blastocysts will as yet be unknown, CGH we will be performed on PB-I specimens from their oocytes to determine their ploidy at a later date. No blastocysts known to be derived from aneuploid oocytes will be transferred. All blastocysts derived from euploid oocytes will be cryopreserved and stored for the recipients to whom they were originally assigned, for future dispensation.

Twelve consenting oocyte donors will be recruited for generating the oocytes to be tested for competence as described above, is envisaged. The resulting euploid blastocysts will subsequently be transferred to the uteri of up to 25 selected embryo recipients (as described above).

With this design, the inventors will achieve the following objectives: (i) Determine the practicability of oocyte cryopreservation when confined to euploid oocytes; (ii) Evaluate the effect of oocyte cryopreservation (vitrification), and subsequent thawing, on thawed oocyte viability and the potential for ensuing normal embryogenesis. It will also allow the assessment of fertilization rates of post-ICSI, blastocyst implantation potential and viable pregnancy rate (>8 weeks); (iii) Re-confirm the findings in the Examples 6 and 7 and in so doing confirm the value of PB-I biopsy with CGH in predicting embryo competence as determined by embryo implantation and viable pregnancy generation; and (iv) Demonstrate the effect (if any), of the actual process of cryopreservation and PB-I biopsy and on oocyte and embryo competence.

The anticipated outcomes are that the successful assessment of oocyte and embryo competence will have a positive impact on clinical practice in IVF.

The most immediate impact is expected to be increased viable, euploid pregnancy rates in IVF with the selective fertilization of only competent oocytes.

Fewer embryos will need to be transferred with IVF without compromising pregnancy outcomes thereby drastically reducing the overall incidence of IVF multiple gestations and virtually eliminate high order multiple pregnancies (HOMP-triplets or greater).

This technology could have a very positive impact in countries where the number of oocytes fertilized and/or the number of embryos transferred is restricted by legislation.

Only high-quality, competent embryos from IVF would be selected and cryopreserved for future use.

What is claimed is:

1. A method of selecting a euploid oocyte comprising:
a. harvesting at least one oocyte from a female;
b. isolating a first polar body associated with the at least one oocyte;
c. analyzing the genetic component of the first polar body to obtain a genetic analysis parameter;
c. identifying a euploid oocyte by determining if the genetic analysis parameter is indicative of oocyte euploidy; and
d. selecting the euploid oocyte.

2. The method of claim 1 wherein the analyzing is done by comparative genomic hybridization (CGH).

3. The method of claim 2 wherein the genetic analysis parameter indicative of oocyte euploidy is from about 0.8:1 to about 1.2:1.

4. The method of claim 2 wherein the genetic analysis parameter indicative of oocyte euploidy is from about 0.9:1 to about 1.1:1.

5. The method of claim 2 wherein the genetic analysis parameter indicative of oocyte euploidy is about 1:1.

6. The method of claim 1 wherein the selected euploid oocyte is frozen and stored for a period of time.

7. The method of claim 1 wherein the selected euploid oocyte is fertilized with euploid sperm.

8. The method of claim 7 wherein the fertilized oocyte is cultured to obtain a euploid embryo.

9. The method of claim 7 wherein the embryo is frozen and stored for a period of time.

10. The method of claim 8 wherein the embryo is competent.

11. The method of claim 7 wherein the embryo is transferred to a recipient female.

12. A method of determining the competence of an embryo comprising:
a. harvesting at least one oocyte from a female;
b. isolating a first polar body associated with the at least one oocyte;
c. analyzing the genome of the first polar body to obtain a genetic analysis parameter;
d. correlating the genetic analysis parameter with the ploidy of the oocyte;
c. selecting and fertilizing the oocyte with euploid sperm to obtain an embryo;
d. determining that the embryo is competent if the genetic analysis parameter of the first polar body associated with the oocyte from which the embryo was derived, is indicative of euploidy.

13. The method of claim 12 wherein the analyzing is done by comparative genomic hybridization (CGH).

14. The method of claim 13 wherein the genetic analysis parameter indicative of oocyte euploidy is from about 0.8:1 to about 1.2:1.

15. The method of claim 13 wherein the genetic analysis parameter indicative of oocyte euploidy is from about 0.9:1 to about 1.1:1.

16. The method of claim 13 wherein the genetic analysis parameter indicative of oocyte euploidy is about 1:1.

17. The method of claim 12 wherein the at least one oocyte is frozen and stored for a period of time prior to fertilization.

18. The method of claim 12 wherein the embryo is frozen and stored for a period of time.

19. A method of impregnating a recipient female comprising:
a. harvesting at least one oocyte from a donor female;
b. isolating a first polar body associated with the at least one oocyte;
c. analyzing the genome of the first polar body to obtain a genetic analysis parameter;
d. correlating the genetic analysis parameter with the euploidy of the oocyte;
e. selecting and fertilizing the at least one oocyte with euploid sperm to a zygote;
f. culturing the zygote to obtain an embryo;
g. determining that the embryo is competent if the genetic analysis parameter of the first polar body associated with the oocyte from which the embryo was derived, is indicative of euploidy and
h. transferring the embryo to the female recipient if it was determined to be competent.

20. The method of claim 19 wherein the recipient female and the donor female are the same individual.

21. The method of claim 19 wherein the recipient female and the donor female are different individuals.

22. The method of claim 19 wherein the analyzing is done by comparative genomic hybridization (CGH).

23. The method of claim 22 wherein the genetic analysis parameter indicative of oocyte euploidy is from about 0.8:1 to about 1.2:1.

24. The method of claim 22 wherein the genetic analysis parameter indicative of oocyte euploidyis from about 0.9:1 to about 1.1:1.

25. The method of claim 22 wherein the genetic analysis parameter indicative of oocyte euploidy is about 1:1.

26. The method of claim 19 wherein the oocyte is frozen and thawed prior to fertilization.

27. The method of claim 19 wherein the embryo is frozen, stored for a period of time and unfrozen prior to transfer to recipient female, if it was determined to be competent.

28. A method of identifying a candidate fertility drug comprising:
a. Harvesting a first plurality of oocytes from a female;
b. isolating a first polar body associated with each of the plurality of oocytes;
c. analyzing the genome of each first polar body to obtain a genetic analysis parameter for each ooctye;
d. identifying a euploid oocyte by determining if its genetic analysis parameter is indicative of oocyte euploidy;
e. comparing the number of euploid oocytes obtained in (d) to the number of oocytes obtained in (a) to obtain a baseline euploid ratio;
f. administering to the female a test compound;
g. harvesting a second plurality of oocytes from the female
h. analyzing the genome of first polar bodies associated with each of the second plurality of oocytes;
i. identifying euploid oocytes of the second plurality of oocytes by determining if the genetic analysis parameters in (h) are indicative of oocyte euploidy;
j. comparing the number of euploid oocytes identified in (i) to the number of oocytes obtained in (g) to obtain a test euploid ratio; and
k. identifying a candidate fertility drug if the administration correlates with an increase in the test euploid ratio compared to the baseline euploid ratio.

29. The method of claim 28 wherein the female is a human.

30. The method of claim 28 wherein the analyzing is done by comparative genomic hybridization (CGH).

31. The method of claim 30 wherein the genetic analysis parameter indicative of oocyte euploidy is from about 0.8:1 to about 1.2:1.

32. The method of claim 30 wherein the genetic analysis parameter indicative of oocyte euploidy is from about 0.9:1 to about 1.1:1.

33. The method of claim 30 wherein the genetic analysis parameter indicative of oocyte euploidy is about 1:1.

34. A method of determining the efficacy of a fertility drug therapeutic regimen on a female comprising:
a. Harvesting a first plurality of oocytes from the female;
b. isolating a plurality of first polar bodies associated with each oocyte;
c. analyzing the genetic component of each first polar body to obtain a genetic analysis parameter for each ooctye;
d. identifying a euploid oocyte by determining if the genetic analysis parameters are indicative of oocyte euploidy;
e. comparing the number of euploid oocytes obtained in (d) to the number of oocytes obtained in (a) to obtain a baseline euploidy ratio;
f. administering to the female a fertility drug therapeutic regimen;
g. harvesting a second plurality of oocytes from the female
h. analyzing the genetic component of each first polar body to obtain a test genetic analysis parameter for each ooctye harvested in the second plurality of oocytes;
i. identifying euploid oocytes in the second plurality of oocytes by determining if the test genetic analysis parameters are indicative of oocyte euploidy;
comparing the number of euploid oocytes identified in (i) to the number of oocytes obtained in (g) to obtain a test euploid ratio; and
k. determining that the fertility drug therapeutic regimen is effective when the administration of the fertility drug therapeutic regimen correlates with an increase in the test euploid ratio compared to the baseline euploid ratio.

35. The method of claim 34 wherein the female is a human.

36. The method of claim 34 wherein the analyzing is done by comparative genomic hybridization (CGH).

37. The method of claim 35 wherein the genetic analysis parameter indicative of oocyte euploidy is from about 0.8:1 to about 1.2:1.

38. The method of claim 35 wherein the genetic analysis parameter indicative of oocyte euploidy is from about 0.9:1 to about 1.1:1.

39. The method of claim 35 wherein the genetic analysis parameter indicative of oocyte euploidy is about 1:1.
